(19) United States
(12) Patent Application Publication
(10) Pub. No.: US 2005/0009078 A1
(43) Pub. Date: Jan. 13, 2005

(54) Method, system, and computer software for providing a genomic web portal

(75) Inventors: David M. Craford, Los Altos, CA (US); Vernon A. Norviel, San Jose, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/903,344

(22) Filed: Jul. 30, 2004

Related U.S. Application Data
(63) Continuation of application No. 10/197,621, filed on Jul. 17, 2002.
Application No. 10/197,621 is a continuation of application No. PCT/US01/02316, filed on Jan. 24, 2001.
(60) Provisional application No. 60/178,077, filed on Jan. 25, 2000.

(51) Int. Cl. C12Q 1/68; G06F 19/00; G01N 33/48; G01N 33/50

(52) U.S. Cl. 435/6; 702/20

(57) ABSTRACT

Systems, methods, and computer program products are described that process inquiries or orders regarding purchase of biological devices, substances, or related reagents. In some implementations, a user selects probe-set identifiers that identify microarray probe sets capable of enabling detection of biological molecules. Corresponding genes or EST's are identified and are correlated with related product data, which is provided to the user. Further, the user may select products for purchase based on the product data. If so, the user's account may be adjusted based on the purchase order. In the same or other implementations, a local genomic database is periodically updated. In response to a user selection of probe-set identifiers, data related to corresponding genes or EST's is provided to the user from the local genomic database.

### RELATED APPLICATION

The present application claims priority from U.S. Provisional Patent Application Ser. No. 60/178,077, entitled “METHOD, SYSTEM, AND COMPUTER SOFTWARE FOR PROVIDING A GENOMIC WEB PORTAL,” filed Jan. 25, 2000, incorporated herein by reference in its entirety for all purposes.

### BACKGROUND

The present invention relates to the field of bioinformatics. In particular, the present invention relates to computer systems, methods, and products for providing genomic information over networks such as the Internet.

Research in molecular biology, biochemistry, and many related health fields increasingly requires organization and analysis of complex data generated by new experimental techniques. These tasks are addressed by the rapidly evolving field of bioinformatics. See, e.g., H. Rashidi and K. Buehler, Bioinformatics Basics: Applications in Biological Science and Medicine (CRC Press, London, 2000); Bioinformatics: A Practical Guide to the Analysis of Gene and Proteins (B. F. Ouelette and A. D. Bzevanis, eds., Wiley & Sons, Inc., 1998), both of which are hereby incorporated herein by reference in their entireties. Broadly, one area of bioinformatics applies computational techniques to large genomic databases, often distributed over and accessed through networks such as the Internet, for the purpose of illuminating relationships among gene structure and/or location, protein function, and metabolic processes.

### SUMMARY OF THE INVENTION

The expanding use of microarray technology is one of the forces driving the development of bioinformatics. In articular, microarrays and associated instrumentation and computer systems have been developed for rapid and large-scale collection of data about the expression of genes or expressed sequence tags (EST's) in tissue samples. The data may be used, among other things, to study genetic characteristics and to detect mutations relevant to genetic and other diseases or conditions. More specifically, the data gained through microarray experiments is valuable to researchers because, among other reasons, many disease states can potentially be characterized by differences in the expression levels of various genes, either through changes in the copy number of the genetic DNA or through changes in levels of transcription (e.g., through control of initiation, provision of RNA precursors, or RNA processing) of particular genes. Thus, for example, researchers use microarrays to answer questions such as: Which genes are expressed in cells of a malignant tumor but not expressed in either healthy tissue or tissue treated according to a particular regime? Which genes or EST's are expressed in particular organs but not in others? Which genes or EST's are expressed in particular species but not in others? Data collection is only an initial step, however, in answering these and other questions. Researchers are increasingly challenged to extract biologically meaningful information from the vast amounts of data generated by microarray technologies, and to design follow-on experiments. A need exists to provide researchers with improved tools and information to perform these tasks.

Systems, methods, and computer program products are described herein to address these and other needs. In some implementations, a web portal processes inquiries or orders regarding purchase of biological devices or substances, or related reagents. The user selects “probe-set identifiers” (a broad term that is described below) that may be associated with probe sets of one or more probes. These probe sets are capable of enabling detection of biological molecules. These biological molecules include, but are not limited to, nucleic acids including DNA representations or mRNA transcripts and/or representations of corresponding genes (such nucleic acids are hereafter, for convenience, referred to simply as “mRNA transcripts”). The corresponding genes or EST's are identified and are correlated with related data, which is provided to the user. In some aspects, the user may select products for purchase based on the data. If the user decides to make a purchase, the user's account may be adjusted based on the purchase order.

An advantage of some of these implementations is that a user may be presented with product suggestions for follow-up experiments based on results from an initial experiment. These initial results are represented by the user's selection of probe-set identifiers by, for example, designating those probe-set identifiers corresponding to probes indicating a relatively high degree of differential expression in control and experimental samples.

In the same or other implementations, a local genomic database is periodically updated. In some aspects, this updating may be made from remote databases. In response to a user selection of probe-set identifiers, data related to genes or EST's are provided to the user from the local genomic database. In other aspects, data related to genes or EST's are provided to the user from the local genomic database in response to a user selection of gene and/or EST identifiers.

Advantages of some of these implementations include the ability of the user to initiate a data request based on the results of experiments. As only one example, the user may indicate these results by selecting probe-set identifiers corresponding to relatively high differential gene expression. These implementations may also be advantageous because the genomic data is locally available at the time of the user's request and generally need not involve the querying of a remote database in response to the user's request. Rather, the querying of remote databases is done periodically as, for example, weekly. Thus, even if the user's selection involves numerous probe-set identifiers indicative of the expression or differential expression of numerous genes or EST's, a response may be provided rapidly to the user from the local genomic database. Significant delays due to multiple or batch interrogations of remote databases are thus generally avoided.

Also, in the preceding or other implementations, a method is described by which a user places a computer-implemented inquiry or order regarding purchase of one or more products. The user selects a first set of probe-set identifiers, and this selection is sent over the Internet to a portal system capable of correlating data with one or more genes or EST's corresponding to the probe sets identified by the user-selected probe-set identifiers. The user receives the correlated data from the portal system. The user may select some or all of the data or otherwise indicate a desire to purchase products related to the data. If the user elects to purchase a product, the user's account may be adjusted accordingly.

In some implementations a system is described for providing data related to one or more genes or EST's, wherein each gene or EST has at least one corresponding probe set identified by a probe-set identifier and capable of enabling detection of a biological molecule. The biological molecule may be a nucleic acid or an mRNA transcript of a corresponding gene. As noted above, one or more of the probe-set identifiers may include a gene or EST identifier, such as an accession number. The system includes an input manager that receives a user selection of a first set of probe-set identifiers; a gene determiner that identifies genes or EST's corresponding to the probe sets identified by the first set of probe-set identifiers; a correlator that correlates the genes or EST's with data; and an output manager that provides the data to the user. The input and output managers of these implementations may be coupled to the user via the Internet.

The first set of probe-set identifiers may be a subset of a second set of probe-set identifiers of probe sets that have enabled detection of the expression or differential expression of their corresponding genes or EST's. For example, the user may have selected the subset using a graphical user interface provided by a probe-array software application. This selection may be made, for instance, by drawing a loop around out-liers in a scatter plot representation of probe sets, where the out-liers indicate probe sets having a relatively high degree of differential expression. As another of many possible examples, the user may select the subset by highlighting entries of probe-set identifiers in an ordered table.

The probe sets typically are disposed on one or more probe arrays that, as noted, may be any of various types of microarrays such as those synthesized using VLSIPS™ technology (described below) or spotted arrays. Thus, the term “probe set” generally will be understood to include not only a set of synthesized probes in accordance, for example, with VLSIPS™ technology, but also one or more spots as deposited in accordance with various spotted array technologies (also described below). The spots may, as one example, be oligonucleotides or in another be cDNA clones or PCR products generated from those clones. The data may include product data about the availability, pricing, composition, suitability, or ordering of various products including a biological device or substance, or a reagent that may be used with a biological device or substance or additional information such as nucleotide or protein sequence information or locational or functional annotation information. As some examples, the device may be a probe array or a microscope slide, or the substance may be a clone, oligonucleotide, antibody, or protein.

Other implementations are directed to methods for providing data related to one or more genes or EST's, wherein each gene or EST has at least one corresponding probe set identified by a probe-set identifier and capable of enabling detection of a biological molecule. The biological molecule may be a nucleic acid or an mRNA transcript of a corresponding gene. The method includes the steps of: receiving a user selection of a first set of probe-set identifiers; identifying genes or EST's corresponding to the probe sets identified by the first set of probe-set identifiers; correlating the genes or EST's with data; and providing the data to the user. Yet other implementations are directed to a computer program product that implements the preceding methods.

Further implementations are directed to a method for placing a computer-implemented inquiry or order regarding purchase of one or more products. This method includes the steps of: receiving at a user computer a user selection of a first set of one or more probe-set identifiers, wherein each probe-set identifier identifies a probe set that has enabled detection of the expression of a corresponding gene; providing the user selection over the Internet to a portal system capable of correlating data with one or more genes or EST's corresponding to the probe sets identified by the first set of probe-set identifiers; and receiving the correlated data from the portal system. The user may also select product data for purchase.

Yet another implementation is directed to a system for providing data related to one or more genes or EST's, wherein each gene or EST has at least one corresponding probe set identified by a probe-set identifier and capable of enabling detection of a biological molecule. The biological molecule may be a nucleic acid or an RN A transcript of a corresponding gene. The system includes a database manager that periodically updates a local genomic database comprising data related to the genes or EST'S; an input manager that receives a user selection of probe-set identifiers; a user-service manager that constructs from the local genomic database data related to genes or EST's corresponding to the probe-set identifiers; and an output manager that provides the data to the user.

In the preceding implementations, the database manager may periodically update the local genomic database, for example, weekly, with sequence data, exonic structure or location data, splice-variants data, marker structure or location data, polymorphism data, homology data, protein-family classification data, pathway data, alternative-gene naming data, literature-recitation data, annotation data, other genomic or proteomic data, or any combination thereof. This updating may be accomplished by periodic communication with remote databases, possibly over the Internet. Any of hundreds of public or proprietary remote databases may be included, such as GenBank, GenBank New, SwissProt, GenPept, DB EST, Unigene, PIR, Prosite, PFAM, Prodom, Blocks, PDB, PDBfinder, EC Enzyme, Kegg Pathway, Kegg Ligand, OMIM, OMIM Map, OMIM Allele, DB SNP, and/or PubMed. Whereas the database manager periodically communicates with remote databases, typically (but not necessarily) not in response to a user's request, the input manager typically (but not necessarily) dynamically receives the user's selection of probe-set identifiers. The word “dynamically,” as used in this context is intended to indicate an essentially real-time response to a user inquiry.

In yet further implementations, a system is described for providing product data, which may include biological product data. The system has an input manager that receives from a user a gene, EST, and/or probe-set identifier. For example, the user may specify one or more gene accession numbers. The system also has a user-service manager that correlates or associates the gene, EST, and/or probe-set identifier with one or more product data. The user-service manager further causes, optionally in cooperation with a database manager, the product data to be obtained from one or more local and/or remote databases or other local or remote source of data, e.g., a web page. Also included in the system is an output manager that provides the product data to the user. In some aspects, a User account may be adjusted based on the purchase, or a vendor account may be adjusted for referring the user to the vendor. The receipt of information from, and provision of information to, the user may be done over a network, such as the Internet. In other aspects, a method is described for providing product data, e.g., biological product data. The method includes the steps of: receiving from a user a gene, EST, and/or probe-set identifier; correlating the gene, EST, and/or probe-set identifier with one or more product data; causing the product data to be obtained from a local and/or a remote database or other local and/or remote source of data; and providing the product data to the user. The method may optionally include adjusting a user account based on the purchase, or adjusting a vendor account for referring the user to the vendor.

A further aspect is a system for providing product data related to one or more genes or EST's. Each gene or EST has at least one corresponding probe set identified by a probe-set identifier and capable of enabling detection of a biological molecule. The system includes an input manager that receives one or more of the probe-set identifiers; a correlator that correlates the probe-set identifiers with a first set of one or more product data; and an output manager that provides the first set of data to the user. Yet another aspect is a system for providing product data related to one or more genes or EST's. The system includes an input manager that receives one or more gene and/or EST identifiers; a correlator that correlates the identifiers with a first set of one or more product data; and an output manager that provides the first set of data to the user.

An additional aspect is a method for providing product data related to one or more genes or EST's. Each gene or EST has at least one corresponding probe set identified by a probe-set identifier and capable of enabling detection of a biological molecule. The method includes the steps of receiving one or more of the probe-set identifiers; correlating the probe-set identifiers with a first set of one or more product data; and providing the first set of data to the user. Yet another aspect is a method for providing product data related to one or more genes or EST'S. The method includes the steps of receiving one or more gene and/or EST identifiers; correlating the identifiers with a first set of one or more product data; and providing the first set f data to the user.

According to another aspect, a system is described for providing product data related to one or more genes or EST's. The system includes receiving means for receiving one or more gene or EST identifiers over the Internet; correlating means for correlating the gene or EST identifiers with one or more product data; and providing means for providing the product data to the user.

According to yet another aspect, a system is described for providing product data related to one or more genes or EST's, wherein each gene or EST has at least one corresponding probe set identified by a probe-set identifier and capable of enabling detection of a biological molecule. The system includes receiving means for receiving from a user a selection of a first set of one or more of the probe-set identifiers; correlating means for correlating the first set of probe-set identifiers with a first set of one or more product data; and providing means for providing the first set of data to the user.

In an additional aspect, a system is described for providing data related to one or more genes or EST's, wherein each gene or EST has at least one corresponding probe set identified by a probe-set identifier and capable of enabling detection of a biological molecule. The system includes updating means for periodically updating a local genomic database comprising data related to the genes or EST's; input managing means for receiving from a user a selection of a first set of one or more of the probe-set identifiers; data managing means for periodically updating from the local genomic database a first set of data related to genes or EST's corresponding to the first set of probe-set identifiers; and providing means for providing the first set of data to the user.

The above implementations are not necessarily inclusive or exclusive of each other and may be combined in any manner that is non-conflicting and otherwise possible, whether they be presented in association with a same, or a different, aspect or implementation. The description of one implementation is not intended to be limiting with respect to other implementations. Also, any one or more function, step, operation, or technique described elsewhere in this specification may, in alternative implementations, be combined with any one or more function, step, operation, or technique described in the summary. Thus, the above implementations are illustrative rather than limiting.

### BRIEF DESCRIPTION OF THE DRAWINGS

The above and further advantages will be more clearly appreciated from the following detailed description when taken in conjunction with the accompanying drawings. In the drawings, like reference numerals indicate like structures or method steps and the leftmost one or two digits of a reference numeral indicates the number of the figure in which the referenced element first appears (for example, the element 180 appears first in FIG. 1 and element 1020 first appears in FIG. 10). In functional block diagrams, rectangles generally indicate functional elements, parallelograms generally indicate data, rectangles with curved sides generally indicate stored data, rectangles with a pair of double borders generally indicate predefined functional elements, and keystone shapes generally indicate manual operations. In method flow charts, rectangles generally indicate method steps and diamond shapes generally indicate decision elements. All of these conventions, however, are intended to be typical or illustrative, rather than limiting.

FIG. 1 is a functional block diagram of a probe-array analysis system including a scanner and a computer system on which may be executed computer applications suitable for providing probe-set identifiers and for receiving user selections of probe-set identifiers for processing;

FIG. 2 is a functional block diagram of one embodiment of probe-array analysis applications as illustratively stored for execution in system memory of the computer system of FIG. 1;

FIG. 3 is a functional block diagram of a conventional system for obtaining genomic information over the Internet;

FIG. 4 is a functional block diagram of one embodiment of a genomic portal coupled over the Internet to remote databases and web pages and to clients including networks having user computer systems including that of FIG. 1;

FIG. 5 is a functional block diagram of one embodiment of the genomic portal of FIG. 4 including illustrative embodiments of a database server, portal application computer system, and portal-side Internet server;

FIG. 6 is a simplified graphical representation of one embodiment of computer application platforms for implementing the genomic portal of FIGS. 4 and 5 in communication with clients such as those shown in FIG. 4;

FIG. 7 is a flow chart of one embodiment of a method for providing a user with genomic product information related to gene expression, or differential expression, experimental results;

FIG. 8 is a functional block diagram of one embodiment of a user-service manager application as may be executed on the portal application computer system of FIG. 5;

FIG. 9 is a simplified graphical representation of one embodiment of a gene or probe-set identifier to database such as may be by the user-service manager of FIG. 8 in connection with the method of FIG. 7;

FIG. 10 is one embodiment of a graphical user interface that may be generated by a probe-array analysis application of FIG. 2; and

FIG. 11 is another embodiment of a graphical user interface that may be generated by a probe-array analysis application of FIG. 2.

### DETAILED DESCRIPTION

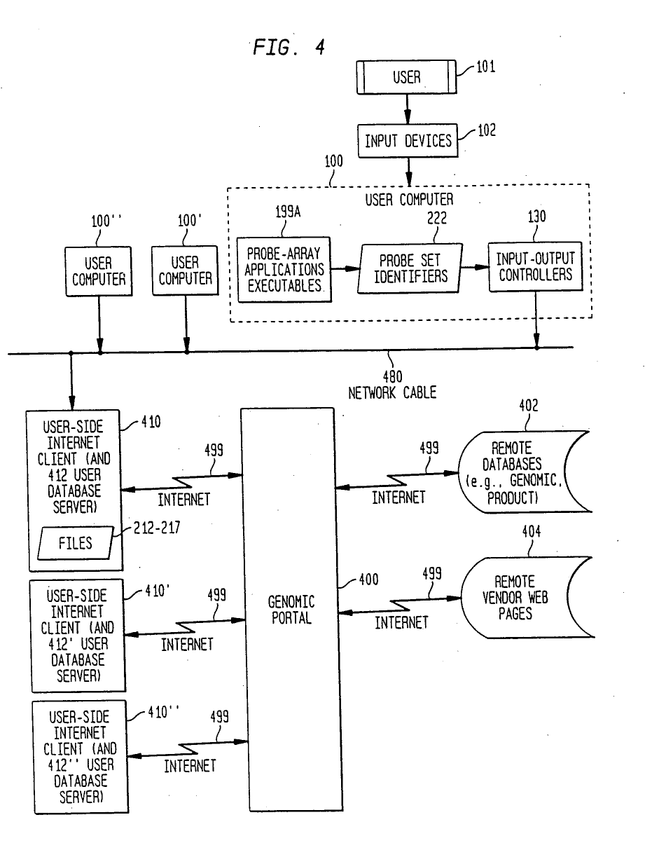

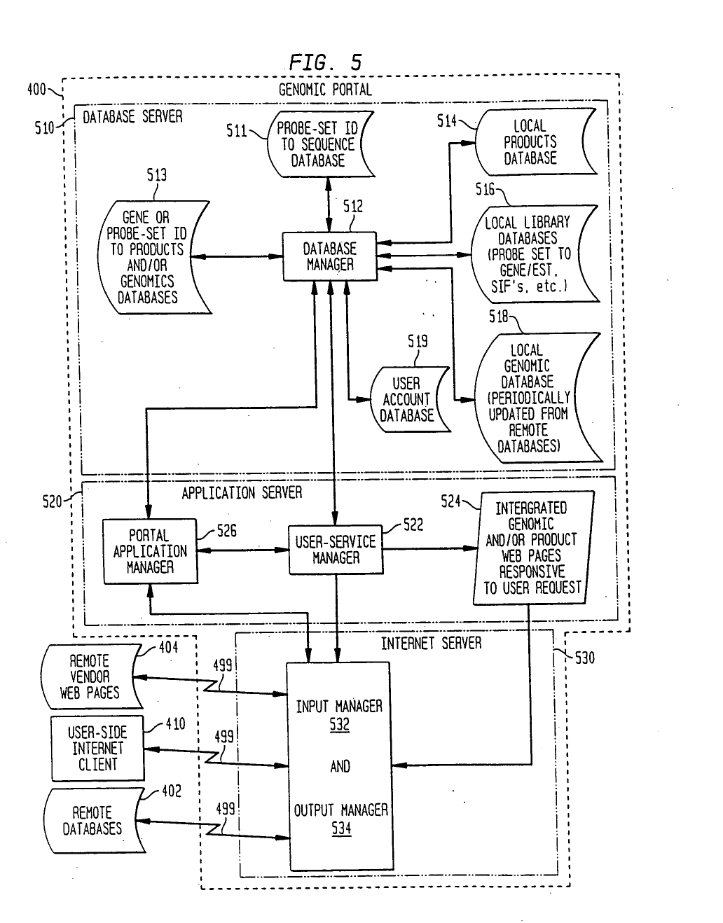

Systems, methods, and computer products are now described with reference to an illustrative embodiment referred to as genomic portal 400. Portal 400 is shown in an Internet environment in FIG. 4, and is illustrated in greater detail in FIGS. 5-11.

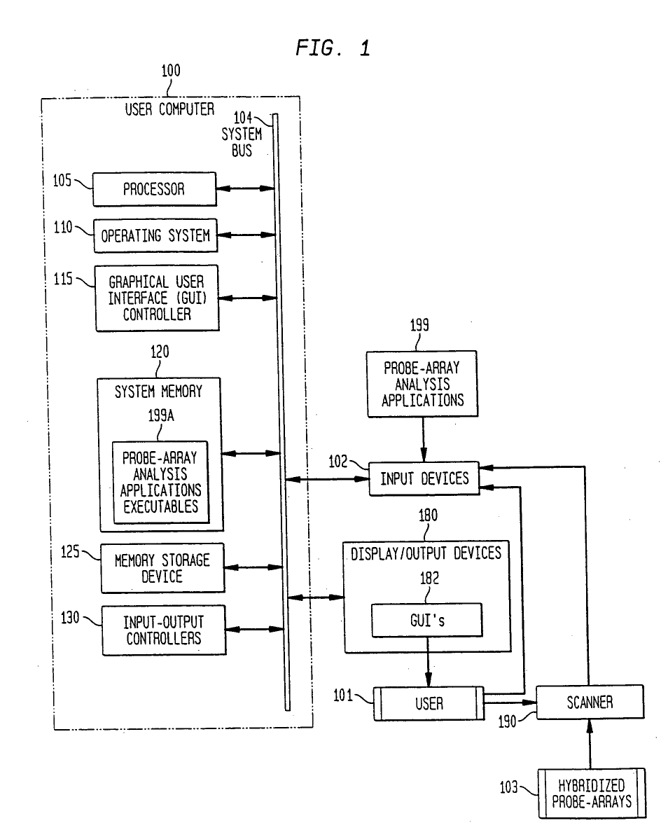

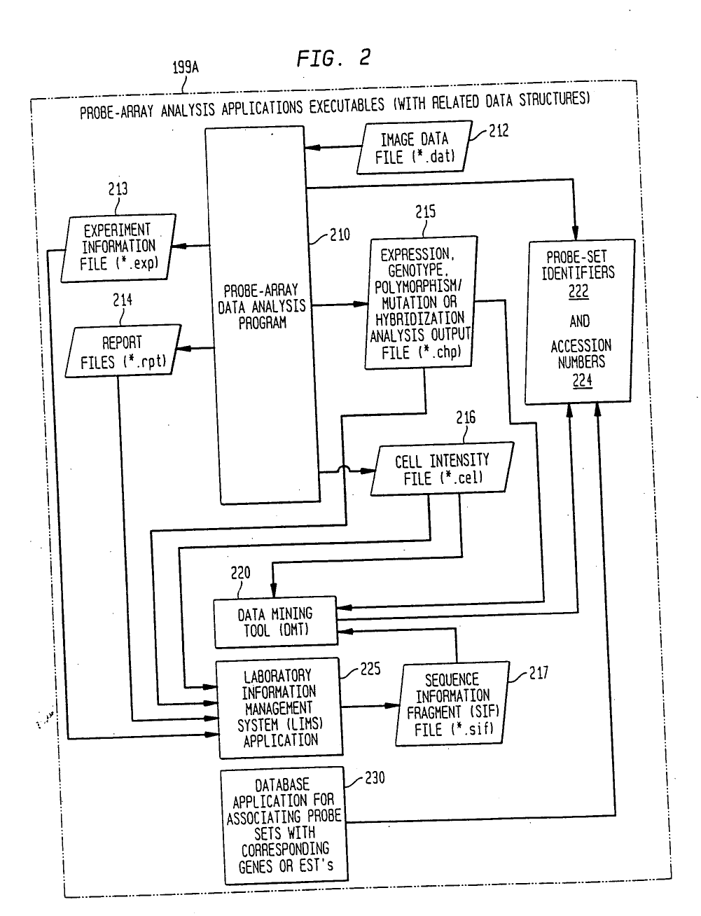

In a typical implementation, portal 400 may be used to provide a user with information related to results from experiments with probe arrays. The experiments often involve the use of scanning equipment to detect hybridization of probe-target pairs, and the analysis of detected hybridization by various software applications, as now described in relation to FIGS. 1 and 2.

### Probe Arrays 103

Various techniques and technologies may be used for depositing or synthesizing dense arrays of biological materials on a substrate or support. For example, Affymetrix® GeneChip® arrays, manufactured by Affymetrix, Inc. of Santa Clara, Calif., are synthesized in accordance with techniques sometimes referred to as VLSIPS™ (Very Large Scale Immobilized Polymer Synthesis) technologies. Some aspects of VLSIPS™ technologies are described in the following U.S. Pat. No. 5,143,854 to Pirrung, et al.; U.S. Pat. No. 5,445,934 to Fodor, et al.; U.S. Pat. No. 5,744,305 to Fodor, et al.; U.S. Pat. No. 5,831,070 to Pease, et al.; U.S. Pat. No. 5,837,832 to Chee, et al.; U.S. Pat. No. 6,022,963 to McGall, et al.; and U.S. Pat. No. 6,083,697 to Beecher, et al. Each of these patents is hereby incorporated by reference in its entirety. The probes of these arrays consist of oligonucleotides, which are synthesized by methods that include the steps of activating regions of a substrate and then contacting the substrate with a selected monomer solution. The regions are activated with a light source shown through a mask in a manner similar to photolithography techniques used in the fabrication of integrated circuits. Other regions of the substrate remain inactive because the mask blocks them from illumination. By repeatedly activating different sets of regions and contacting different monomer solutions with the substrate, a diverse array of polymers is produced on the substrate. Various other steps, such as washing unreacted monomer solution from the substrate, are employed in various implementations of these methods.

These probes typically are used in conjunction with tagged biological samples such as cells, proteins, genes or EST's, other DNA sequences, or other biological elements. These samples, referred to herein as “targets,” are processed so that they are spatially associated with certain probes in the probe array. For example, one or more chemically tagged biological samples, i.e., the targets, are distributed over the probe array. Some targets hybridize with at least partially complementary probes and remain at the probe locations, while non-hybridized targets are washed away. These hybridized targets, with their “tags” or “labels,” are thus spatially associated with the targets' complementary probes. The hybridized probe and target may sometimes be referred to as a “probe-target pair.” Detection of these pairs can serve a variety of purposes, such as to determine whether a target nucleic acid has a nucleotide sequence identical to or different from a specific reference sequence. See, for example, U.S. Pat. No. 5,837,832, referred to and incorporated above. Other uses include gene expression monitoring and evaluation (see, e.g., U.S. Pat. No. 5,800,992 to Fodor, et al.; U.S. Pat. No. 6,040,138 to Lockhart, et al.; and International App. No. PCT/US98/15151, published as WO99/05323, to Balaban, et al.), genotyping (U.S. Pat. No. 5,856,092 to Dale, et al.), or other detection of nucleic acids. The '992, '138, and '092 patents, and publication WO99/05323, are incorporated by reference herein in their entirety for all purposes.

Other techniques exist for depositing probes on a substrate or support. For example, “spotted arrays” are commercially fabricated on microscope slides. These arrays consist of liquid spots containing biological material of potentially varying compositions and concentrations. For instance, a spot in the array may include a few strands of short oligonucleotides in a water solution, or it may include a high concentration of long strands of complex proteins. The Affymetrix® 417™ Arrayer is a device that deposits a densely packed array of biological material on a microscope slide in accordance with these techniques, aspects of which are described in PCT Application No. PCT/US99/00730 (International Publication Number WO 99/36760), hereby incorporated by reference in its entirety. Other echniques for generating spotted arrays also exist. For example, U.S. Pat. No. 6,040,193 to Winkler, et al. is directed to processes for dispensing drops to generate spotted arrays. The '193 patent, and U.S. Pat. No. 5,885,837 to Winkler, also describe the use of micro-channels or micro-grooves on a substrate, or on a block placed on a substrate, to synthesize arrays of biological materials. These patents further describe separating reactive regions of a substrate from each other by inert regions and spotting on the reactive regions. The '193 and '837 patents are hereby incorporated by reference in their entireties. Another technique is based on ejecting jets of biological material to form a spotted array. Other implementations of the jetting technique may use devices such as syringes or piezo electric pumps to propel the biological material. Various other techniques exist for synthesizing, depositing, or positioning biological material onto or within a substrate.

To ensure proper interpretation of the term “probe” as used herein, it is noted that contradictory conventions exist in the relevant literature. The word “probe” is used in some contexts to refer not to the biological material that is synthesized on a substrate or deposited on a slide, as described above, but to what has been referred to herein as the “target.” To avoid confusion, the term “probe” is used herein to refer to probes such as those synthesized according to the VLSIPS™ technology; the biological materials deposited so as to create spotted arrays; and materials synthesized, deposited, or positioned to form arrays according to other current or future technologies. Thus, microarrays formed in accordance with any of these technologies may be referred to generally and collectively hereafter for convenience as “probe arrays.” Moreover, the term “probe” is not limited to probes immobilized in array format. Rather, the functions and methods described are also useful for providing genomic information and intelligent e-commerce for other parallel assay devices. For example, these functions and methods may be applied with respect to probe-set identifiers that identify probes immobilized on or in beads, optical fibers, or other substrates or media.

Probes typically are able to detect the expression of corresponding genes or EST's by detecting the presence or abundance of mRNA transcripts present in the target. This detection may, in turn, be accomplished by detecting labeled cRNA that is derived from cDNA derived from the mRNA in the target. In general, a probe set contains sub-sequences in unique regions of the transcripts and does not correspond to a full gene sequence. The word “set” generally is used herein to refer to one or more; e.g., a probe set may consist of one or more probes, and a set of probe-set identifiers may consist of one or more probe-set identifiers.

### Scanner 190

FIG. 1 is a functional block diagram of a system that is suitable for, among other things, analyzing probe arrays that have been hybridized with labeled targets. Representative hybridized probe arrays 103 of FIG. 1 may include probe arrays of any type, as noted above. Labeled targets in hybridized probe arrays 103 may be detected using various commercial devices, referred to for convenience hereafter as “scanners.” An illustrative device is shown in FIG. 1 as scanner 190. Scanners image the targets by detecting fluorescent or other emissions from the labels, or by detecting transmitted, reflected, or scattered radiation. These processes are generally and collectively referred to hereafter for convenience simply as involving the detection of “emissions.” Various detection schemes are employed depending on the type of emissions and other factors. A typical scheme employs optical and other elements to provide excitation light and to selectively collect the emissions. Also generally included are various light-detector systems employing photodiodes, charge-coupled devices, photomultiplier tubes, or similar devices to register the collected emissions. For example, a scanning system for use with a fluorescent label is described in U.S. Pat. No. 5,143,854, incorporated by reference above. Other scanners or scanning systems are described in U.S. Pat. Nos. 5,578,832; 5,631,734; 5,834,758; 5,981,956 and 6,025,601, and in PCT Application PCT/US99/06097 (published as WO99/47964), each of which is hereby incorporated by reference in its entirety for all purposes.

Scanner 190 provides data representing the intensities (and possibly other characteristics, such as color) of the detected emissions, as well as the locations on the substrate where the emissions were detected. The data typically are stored in a memory device, such as system memory 120 of user computer 100, in the form of a data file. One type of data file, such as image data file 212 shown in FIG. 2, typically includes intensity and location information corresponding to elemental sub-areas of the scanned substrate. The term “elemental” in this context means that the intensities, and/or other characteristics, of the emissions from this area each are represented by a single value. When displayed as an image for viewing or processing, elemental picture elements, or pixels, often represent this information. Thus, for example, a pixel may have a single value representing the intensity of the elemental sub-area of the substrate from which the emissions were scanned. The pixel may also have another value representing another characteristic, such as color. For instance, a scanned elemental sub-area in which high-intensity emissions were detected may be represented by a pixel having high luminance (hereafter, a “bright” pixel), and low-intensity emissions may be represented by a pixel of low luminance (a “dim” pixel). Alternatively, the chromatic value of a pixel may be made to represent the intensity, color, or other characteristic of the detected emissions. Thus, an area of high-intensity emission may be displayed as a red pixel and an area of low-intensity emission as a blue pixel. As another example, detected emissions of one wavelength at a particular sub-area of the substrate may be represented as a red pixel, and emissions of a second wavelength detected at another sub-area may be represented by an adjacent blue pixel. Many other display schemes are known.

### Probe-Array Analysis Applications 199

Generally, a human being may inspect a printed or displayed image constructed from the data in an image file and may identify those cells that are bright or dim, or are otherwise identified by a pixel characteristic (such as color). However, it frequently is desirable to provide this information in an automated, quantifiable, and repeatable way that is compatible with various image processing and/or analysis techniques. For example, the information may be provided for processing by a computer application that associates the locations where hybridized targets were detected with known locations where probes of known identities were synthesized or deposited. Information such as the nucleotide or monomer sequence of target DNA or RNA may then be deduced. Techniques for making these deductions are described, for example, in U.S. Pat. No. 5,733,729 to Lipshutz, which hereby is incorporated by reference in its entirety for all purposes, and in U.S. Pat. No. 5,837,832, noted and incorporated above.

A variety of computer software applications are commercially available for controlling scanners (and other instruments related to the hybridization process, such as hybridization chambers), and for acquiring and processing the image files provided by the scanners. Examples are the Jaguar™ application from Affymetrix, Inc., aspects of which are described in U.S. Provisional Patent Application Ser. No. 60/226,999, filed Aug. 22, 2000, and the Microarray Suite application from Affymetrix, aspects of which are described in U.S. Provisional Patent Application Ser. No. 60/220,587, filed Jul. 25, 2000. The processed image files produced by these applications often are further processed to extract additional data. In particular, data-mining software applications often are used for supplemental identification and analysis of biologically interesting patterns or degrees of hybridization of probe sets. An example of a software application of this type is the Affymetrix® Data Mining Tool. Software applications also are available for storing and managing the enormous amounts of data that often are generated by probe-array experiments and by the image-processing and data-mining software noted above. An example of these data-management software applications is the Affymetrix® Laboratory Information Management System (LIMS), aspects of which are described in U.S. Provisional Patent Application Ser. No. 60/220,645, filed Jul. 25, 2000. In addition, various proprietary databases accessed by database management software, such as the Affymetrix® EASI (Expression Analysis Sequence Information) database and database software, provide researchers with associations between probe sets and gene or EST identifiers. All of the patent applications noted in this paragraph are hereby incorporated herein by reference in their entireties.

For convenience of reference, these types of computer software applications (i.e., for acquiring and processing image files, data mining, data management, and various database and other applications related to probe-array analysis) are generally and collectively represented in FIG. 1 as probe-array analysis applications 199. FIG. 2 is a functional block diagram of probe-array analysis applications 199 as illustratively stored for execution (as executable code 199A corresponding to applications 199) in system memory 120 of user computer 100 of FIG. 1.

As will be appreciated by those skilled in the relevant art, it is not necessary that applications 199 be stored on and/or executed from computer 100; rather, some or all of applications 199 may be stored on and/or executed from an applications server or other computer platform to which computer 100 is connected in a network. For example, it may be particularly advantageous for applications involving the manipulation of large databases, such as Affymetrix® LIMS or Affymetrix® Data Mining Tool (DMT), to be executed from a database server such as user database server 412 of FIG. 4. Alternatively, LIMS, DMT, and/or other applications may be executed from computer 100, but some or all of the databases upon which those applications operate may be stored for common access on server 412 (perhaps together with a database management program, such as the Oracle® 8.0.5 database management system from Oracle Corporation). Such networked arrangements may be implemented in accordance with known techniques using commercially available hardware and software, such as those available for implementing a local-area network or wide-area network. A local network is represented in FIG. 4 by the connection of user computer 100 to user database server 412 (and to user-side Internet client 410, which may be the same computer) via network cable 480. Similarly, scanner 190 (or multiple scanners) may be made available to a network of users over cable 480 both for purposes of controlling scanner 190 and for receiving data input from it.

Referring again to FIG. 2, application executables 199A generate data of various kinds in various formats, of which those shown are only illustrations. For convenience, the term “file” often is used herein to refer to data generated or used by application executables 199A, but any of a variety of alternative techniques known in the relevant art for storing, conveying, and/or manipulating data may be employed. In the example of this figure, data analysis program 210 receives image data file 212 from scanner 190 and generates, among other things, cell intensity file 216. File 216 of this example contains, for each probe scanned by scanner 190, a single value representative of the intensities of pixels measured by scanner 190 for that probe. Thus, this value is a measure of the abundance of tagged mRNA's present in the target that hybridized to the corresponding probe. Many such mRNA's may be present in each probe, as a probe may include, for example, millions of oligonucleotides designed to detect the mRNA's.

In the illustrated example, probe-array data analysis program 210 generates an experiment information file 213 that contains information, often input by user 101, about the experiment, the sample, and the probe array. A principal function of data analysis program 210 of this example is to analyze file 216 and/or file 212, perhaps together with information from file 213 and internal library files (not shown) that specify details regarding the sequences and locations of probes and controls. The goals of programs such as data analysis program 210 of this example is generally to provide information such as the degree of hybridization, absolute and/or differential (over two or more experiments) expression, genotype comparisons, detection of polymorphisms and mutations, and other analytical results. In this example, file 215 represents this analytical output of data analysis program 210. Data analysis program 210 may process file 215 to create report files 214 that may be responsive to requests by user 101 regarding form and content. As will be appreciated by those skilled in the relevant art, the preceding and following descriptions of files, reports, and data representations generated by illustrative data analysis program 210 are exemplary only, and the data described, and other data, may be processed, combined, arranged, and/or presented in many other ways.

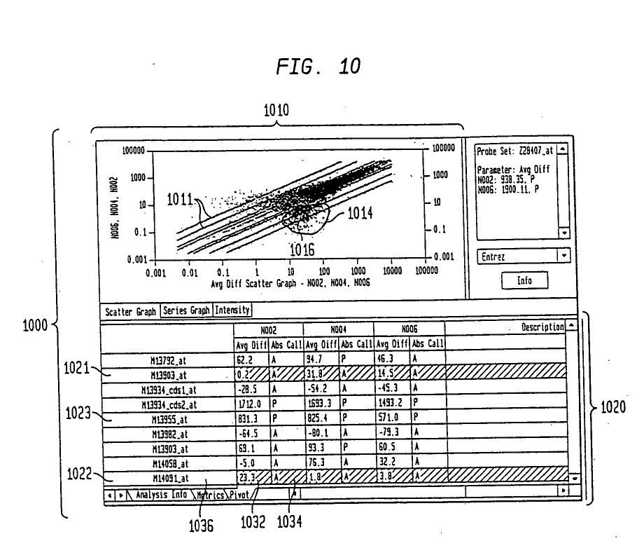

Data analysis program 210 also generates various types of plots, graphs, tables, and other tabular and/or graphical representations of analytical data such as contained in file 215. An illustrative example is shown in FIG. 10, which shows a graphical user interface (GUI) 1000 having scatter plot window 1010 and tabular window 1020. In scatter plot window 1010, lines 1011 provide a reference to the degree of differential expression as measured by probe sets in different experiments. The location of dots, each representing a probe set from one or more microarrays, specifies along one axis the degree of expression of the probe set in one experiment or set of experiments (for example, experiments measuring control samples) and, along the other axis, the degree of expression in another experiment or set of experiments (for example, experiments measuring disease samples).

In FIG. 10, user 101 has drawn line 1014 (using techniques well known in the art) around a cluster of dots 1016. In tabular window 1020, each probe set corresponding to a dot in window 1010 is identified and described in a separate row. In this example, the row entries include a measure of the degree of expression in a particular experiment, as in column 1032, and an indication of whether expression was absent (A) or present (P) in the experiment, as in column 1034. Rows corresponding to dots, i.e., probe sets, encircled in loop 1014 are highlighted in window 1020 so that user 101 may readily identify information about the selected probe sets. In addition, each row in window 1020 includes a probe-set identifier, as in column 1036.

For example, the probe sets corresponding to rows 1021 and 1022 are highlighted to show that their corresponding dots in window 1010 have been encircled. The entries in column 1036 for these rows, i.e., “M13903_at” and “M14091_at,” respectively, are probe-set identifiers for their respective probe sets. FIG. 10 thus is illustrative of numerous techniques by which user 101 may select probe-set identifiers. In particular, user 101 has made these selections in the present example by encircling dots in window 1010 (in which case the selected probe-set identifiers include the encircled dots) and/or by selecting a row in window 1020 (in which case the selected probe-set identifiers include the names in column 1036). Probe-set identifiers 222, as shown in FIG. 2, represent these or other probe-set identifiers that may be provided by applications such as data analysis program 210 for selection by user 101. Also, the convention used in data analysis program 210 of this example for naming probe sets includes information that, in some cases, indicates the accession number of the gene or EST corresponding to the probe set. For example, the probe-set identification name “M13903_at” in row 1021 indicates that the accession number of the gene or EST corresponding to the probe set corresponding to that row is M13903. In other examples, the corresponding accession number may be displayed directly. The provision of these accession numbers for selection by user 101 is represented by accession numbers 124 in FIG. 2. Although, as noted, accession numbers may serve as a type of probe-set identifier (and thus accession numbers 124 may be considered as a subset of probe-set identifiers 222), they are shown distinctly in FIG. 2 for convenience of illustration and discussion.

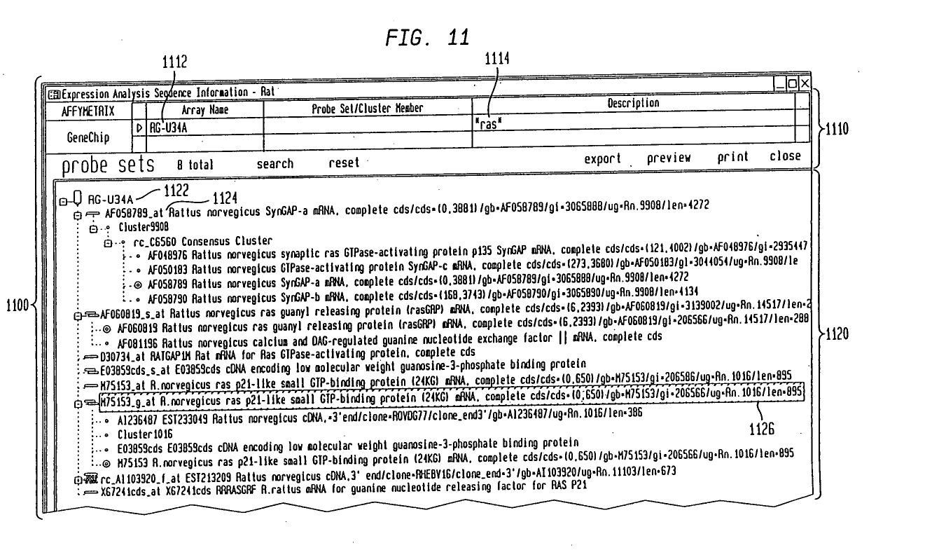

Other of applications executables 199A, such as data mining tool 220, may also provide probe-set identifiers 222 (optionally including accession numbers 224) to user 101. A further example is database application 230, an illustrative GUI of which is represented in FIG. 11. Database application 230 is an application for associating probe sets, typically identified by probe-set identifiers such as names, numbers, and/or symbols, with corresponding genes or EST's. One example of database 230 is the EASI database application from Affymetrix, noted above. In the example of FIG. 11, GUI 1100 includes a query window 1110 and a results window 1120. As shown in FIG. 11, user 101 has effectively created a query, in accordance with known techniques, by selecting a particular probe array 1112 and a portion 1114 of a descriptive text associated with array 1112 or any probe set associated with array 1112. Application 230 conducts a search of its database (not shown) and displays the results of the query in window 1120. As noted below with respect to database FIG. 5, the functions of database application 230 and its associated database may also, or alternatively, be included in portal 400 so that the user's query is satisfied by interrogation of local library databases 516 by database manager 512. In either case, the results of the user's query typically include identification of probe arrays, such as array 1122, and probe-set identifiers, such as identifiers 1124 and 1126, that satisfy the query. As in the previous example, the name given to identifier 1124, “AF058789_at,” may be indicative of the accession number of the gene or EST corresponding to the probe set that it identifies. User 101 may highlight a probe-set identifier such as is shown in FIG. 11 with respect to identifier 1126. The well known tree structure of window 1120 indicates that the probe set identified by identifier 1126 is disposed on array 1122. Descriptive information related to the probe set identified by identifier 1126 is also highlighted and displayed in the same row of the tree structure as identifier 1126.

LIMS application 225 is also shown in FIG. 2 as an exemplary one of analysis applications executables 199A. Application 225 may manage files used or generated by data analysis program 210 (e.g., files 212-216) as well as files or data generated or used by DMT 220 and other types of probe-array analysis applications. LIMS 225 may store, maintain, process, and display this and other data generated by one or more experimenters over time to facilitate the management and planning of experiments and report on their results. LIMS 225 also may provide, based on a library database (not shown), SIF information represented in FIG. 2 by file 217 (and described below). As noted above with respect to application 230, file 217 may alternatively, or in addition, be stored and maintained by portal 400. For example, SIF information may be stored in local library databases 516 and managed by database manager 512, which may include a LIMS such as LIMS 225 or incorporate some or all of its functions.

### User Computer 100

User computer 100, shown in FIG. 1, may be a computing device specially designed and configured to support and execute some or all of the functions of probe array applications 199. Computer 100 also may be any of a variety of types of general-purpose computers such as a personal computer, network server, workstation, or other computer platform now or later developed. Computer 100 typically includes known components such as a processor 105, an operating system 110, a graphical user interface (GUI) controller 115, a system memory 120, memory storage devices 125, and input-output controllers 130. It will be understood by those skilled in the relevant art that there are many possible configurations of the components of computer 100 and that some components that may typically be included in computer 100 are not shown, such as cache memory, a data backup unit, and many other devices. Processor 105 may be a commercially available processor such as a Pentium® processor made by Intel Corporation, a SPARC® processor made by Sun Microsystems, or it may be one of other processors that are or will become available. Processor 105 executes operating system 110, which may be, for example, a Windows®-type operating system (such as Windows NT® 4.0 with SP6a) from the Microsoft Corporation; a Unix® or Linux-type operating system available from many vendors; another or a future operating system; or some combination thereof. Operating system 110 interfaces with firmware and hardware in a well-known manner, and facilitates processor 105 in coordinating and executing the functions of various computer programs that may be written in a variety of programming languages. Operating system 110, typically in cooperation with processor 105, coordinates and executes functions of the other components of computer 100. Operating system 110 also provides scheduling, input-output control, file and data management, memory management, and communication control and related services, all in accordance with known techniques.

System memory 120 may be any of a variety of known or future memory storage devices. Examples include any commonly available random access memory (RAM), magnetic medium such as a resident hard disk or tape, an optical medium such as a read and write compact disc, or other memory storage device. Memory storage device 125 may be any of a variety of known or future devices, including a compact disk drive, a tape drive, a removable hard disk drive, or a diskette drive. Such types of memory storage device 125 typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory 120 and/or the program storage device used in conjunction with memory storage device 125.

In some embodiments, a computer program product is described comprising a computer usable medium having control logic (computer software program, including program code) stored therein. The control logic, when executed by processor 105, causes processor 105 to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers 130 could include any of a variety of known devices for accepting and processing information from a user, whether a human or a machine, whether local or remote. Such devices include, for example, modem cards, network interface cards, sound cards, or other types of controllers for any of a variety of known input devices 102. Output controllers of input-output controllers 130 could include controllers for any of a variety of known display devices 180 for presenting information to a user, whether a human or a machine, whether local or remote. If one of display devices 180 provides visual information, this information typically may be logically and/or physically organized as an array of picture elements, sometimes referred to as pixels. Graphical user interface (GUI) controller 115 may comprise any of a variety of known or future software programs for providing graphical input and output interfaces between computer 100 and user 101, and for processing user inputs. In the illustrated embodiment, the functional elements of computer 100 communicate with each other via system bus 104. Some of these communications may be accomplished in alternative embodiments using network or other types of remote communications.

As will be evident to those skilled in the relevant art, applications 199, if implemented in software, may be loaded into system memory 120 and/or memory storage device 125 through one of input devices 102. All or portions of applications 199 may also reside in a read-only memory or similar device of memory storage device 125, such devices not requiring that applications 199 first be loaded through input devices 102. It will be understood by those skilled in the relevant art that applications 199, or portions of it, may be loaded by processor 105 in a known manner into system memory 120, or cache memory (not shown), or both, as advantageous for execution.

### Conventional Techniques for Obtaining Genomic Data

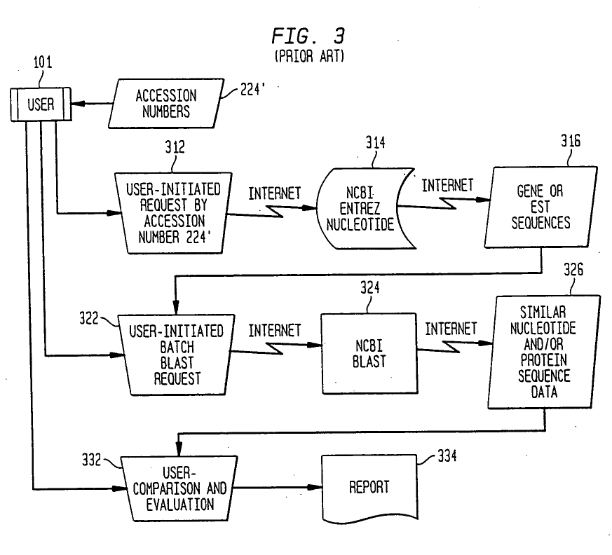

A number of conventional approaches for obtaining genomic data over the Internet are available, some of which are described in the book edited by Ouelette and Bzevanis, incorporated by reference above. FIG. 3 is a functional block diagram representing one simplified example. As shown in FIG. 3, user 101 may consult any of a number of public or other sources to obtain accession numbers 224′. As represented by manual operation 312, user 101 initiates request 312 by accessing through any web browser the Internet web site of the National Center for Biotechnology Information (NCBI) of the National Library of Medicine and the National Institutes of Health (as of January 2001, accessible at the Internet URL http://www.ncbi.nlm.nih.gov/). In particular, user 101 may access the Entrez search and retrieval system that provides information from various databases at NCBI. These databases provide information regarding nucleotide sequences, protein sequences, macromolecular structures, whole genomes, and publication data related thereto. It is illustratively assumed that user 101 accesses in this manner NCBI Entrez nucleotide database 314 and receives information including gene or EST sequences 316. Particularly if accession numbers 224′ represents a large number (e.g., one hundred) of EST's or genes of interest, as may easily be the case following analysis of probe array experiments, the tasks thus far described may take significant time, perhaps hours.

User 101 typically copies sequence information from sequences 316 and pastes this information into an HTML document accessible through NCBI's BLAST web pages 324 (as of January 2001, accessible at http://www.ncbi.nlm.nih.gov/BLAST/). This operation, which also may be time consuming and tedious if many sequences are involved, is represented by user-initiated batch BLAST request 322 of FIG. 3. BLAST is an acronym for Basic Local Alignment Search Tool, and, as is well known in the art, consists of similarity search programs that interrogate sequence databases for both protein and DNA using heuristic algorithms to seek local alignments. For example, user 101 may conduct a BLAST search using the “blastn” nucleotide sequence database. Results of this batch BLAST search, represented by similar nucleotide and/or protein sequence data 326, may not be available to user 101 for many hours. User 101 may then initiate comparisons and evaluations 332, which may be conducted manually or using various software tools. User 101 may subsequently issue report 334 interpreting the findings of the searches and positing strategies and requirements for follow-on experiments.

### Inputs to Genomic Portal 400 from User 101

FIG. 4 is a functional block diagram showing an illustrative configuration by which user 101 may connect with genomic web portal 400. It will be understood that FIG. 4 is simplified and is illustratively only, and that many implementations and variations of the network and Internet connections shown in FIG. 4 will be evident to those of ordinary skill in the relevant art.

User 101 employs user computer 100 and analysis applications 199 as noted above, including generating and/or accessing some or all of files 212-217. As shown in FIG. 4, files 212-217 are maintained in this example on user database server 412 to which user computer 100 is coupled via network cable 480. Computers 100′, 100″, and computers of other users in a local or wide-area network including an Intranet, the Internet, or any other network may also be coupled to server 412 via cable 480. It will be understood that cable 400 is merely representative of any type of network connectivity, which may involve cables, transmitters, relay stations, network servers, and many other components not shown but evident to those of ordinary skill in the relevant art. Via user computer 100, user 101 may operate a web browser served by user-side Internet client 410 to communicate via Internet 499 with portal 400. Portal 400 may similarly be in communication over Internet 499 with other users and/or networks of users, as indicated by Internet clients 410′ and 410″.

As previously noted, the information provided by user 101 to portal 400 typically includes one or more “probe-set identifiers.” These probe-set identifiers typically come to the attention of user 101 as a result of experiments conducted on probe arrays. For example, user 101 may select probe-set identifiers that identify microarray probe sets capable of enabling detection of the expression of mRNA transcripts from corresponding genes or EST's of particular interest. As is well known in the relevant art, an EST is a fragment of a gene sequence that may not be fully characterized, whereas a gene sequence generally is complete and fully characterized. The word “gene” is used generally herein to refer both to full size genes of known sequence and to computationally predicted genes. In some implementations, the specific sequences detected by the arrays that represent these genes or EST's may be referred to as, “sequence information fragments (SIF's)” and may be recorded in a “SIF file,” as noted above with respect to the operations of LIMS 225. In particular implementations, a SIF is a portion of a consensus sequence that has been deemed to best represent the mRNA transcript from a given gene or EST. The consensus sequence may have been derived by comparing and clustering EST's, and possibly also by comparing the EST's to genomic sequence information. A SIF is a portion of the consensus sequence for which probes on the array are specifically designed. With respect to the operations of web portal 400, it is assumed that some microarray probe sets may be designed to detect the expression of genes based upon sequences of EST's.

As was described above, the term “probe set” generally refers to one or more probes from an array of probes on a microarray. For example, in an Affymetrix® GeneChip® probe array, in which probes are synthesized on a substrate, a probe set may consist of 30 or 40 probes, half of which typically are controls. These probes collectively, or in various combinations of some or all of them, are deemed to be indicative of the expression of a gene or EST. In a spotted probe array, one or more spots may similarly constitute a “probe set.”

The term “probe-set identifiers” is used broadly herein in that a number of types of such identifiers are possible and are intended to be included within the meaning of this term. One type of probe-set identifier is a name, number, or other symbol that is assigned for the purpose of identifying a probe set. This name, number, or symbol may be arbitrarily assigned to the probe set by, for example, the manufacturer of the probe array. A user may select this type of probe-set identifier by, for example, highlighting or typing the name. Another type of probe-set identifier as intended herein is a graphical representation of a probe set. For example, dots may be displayed on a scatter plot or other diagram wherein each dot represents a probe set. Typically, the dot's placement on the plot represents the intensity of the signal from hybridized, tagged, targets (as described in greater detail below) in one or more experiments. In these cases, a user may select a probe-set identifier by clicking on, drawing a loop around, or otherwise selecting one or more of the dots. Examples of such selections were provided above in connection with the operations of data analysis program 210 and, more specifically, with respect to user 101 drawing loop 1014 around dots on a scatter plot, and/or selecting a name or accession number associated with highlighting row 1021 or 1022. Other examples were provided above with respect to the selection by user 101 of row 1126 in the database that correlates probe sets with accession numbers and other genomic information.

Yet another type of probe-set identifier, as that term is used herein, includes a nucleotide sequence. For example, it is illustratively assumed that a particular SIF is a unique sequence of 500 bases that is a portion of a consensus sequence or exemplar sequence gleaned from EST and/or genomic sequence information. It further is assumed that one or more probe sets are designed to represent the SIP. A user who specifies all or part of the 500-base sequence thus may be considered to have specified all or some of the corresponding probe sets. As a further example, a user may specify a portion of the 500-base sequence, which may be unique to that SIF, or may also identify another SIF, EST, cluster of EST's, consensus sequence, and/or gene. In that case, the user has specified a probe-set identifier for one or more genes or EST's. In another variation, it is illustratively assumed that a particular SIF is a portion of a particular consensus sequence. It is further assumed that a user specifies a portion of the consensus sequence that is not included in the SIF but that is unique to the consensus sequence or the gene or EST's the consensus sequence is intended to represent. In that case, the sequence specified by the user is a probe-set identifier that identifies the probe set corresponding to the SIF, even though the user-specified sequence is not included in the SIP. Parallel cases are possible with respect to user specifications of partial sequences of EST's and genes or EST's, as those skilled in the relevant art will now appreciate.

A further example of a probe-set identifier is an accession number of a gene or EST. Gene and EST accession numbers are publicly available. A probe set may therefore be identified by the accession number or numbers of one or more EST's and/or genes corresponding to the probe set. The correspondence between a probe set and EST's or genes may be maintained in a suitable database, such as that accessed by database application 230 or local library databases 516, from which the correspondence may be provided to the user. Similarly, gene fragments or sequences other than EST's may be mapped (e.g., by reference to a suitable database) to corresponding genes or EST's for the purpose of using their publicly available accession numbers as probe-set identifiers. For example, a user may be interested in product or genomic information related to a particular SIF that is derived from EST-1 and EST-2. The user may be provided with the correspondence between that SIF (or part or all of the sequence of the SIF) and EST-1 or EST-2, or both. To obtain product or genomic data related to the SIF, or a partial sequence of it, the user may select the accession numbers of EST-1, EST-2, or both.

### Genomic Web Portal 400

Genomic web portal 400 provides to user 101 data related to one or more genes or EST's. Each gene or EST has at least one corresponding probe set that is identified by a probe-set identifier that, as just noted, ay be a number, name, accession number, symbol, graphical representation (e.g., dot or highlighted tabular entry), or nucleotide sequence, as illustrative and non-limiting examples. The corresponding probe sets are capable of enabling detection of the expression of their corresponding gene. In response to a user selection of one or more probe-set identifiers, portal 400 provides user 101 with genomic information and/or information regarding biological products. This information may be helpful to user 101 in analyzing the results of experiments and in designing or implementing follow-up experiments.

FIG. 5 is a functional block diagram of one of many possible embodiments of portal 400. In this example, portal 400 has hardware components including three computer platforms: database server 510, Internet server 530, and application server 520. Various functional elements of portal 400, such as database manager 512, input and output managers 532 and 534, and user-service manager 522, carry out their operations on these computer platforms. That is, in a typical implementation, the functions of managers 512, 532, 534, and 522 are carried out by the execution of software applications on and across the computer platforms represented by servers 510, 530, and 520. Portal 400 is described first with respect to its computer platforms, and then with respect to its functional elements.

Each of servers 510, 520 and 530 may be any type of known computer platform or a type to be developed in the future, although they typically will be of a class of computer commonly referred to as servers. However, they may also be a main frame computer, a work station, or other computer type. They may be connected via any known or future type of cabling or other communication system, either networked or otherwise. They may be co-located or they may be physically separated. Various operating systems may be employed on any of the computer platforms, possibly depending on the type and/or make of computer platform chosen. Appropriate operating systems include Windows NT®, Sun Solaris, Linux, OS/400, Compaq Tru64 Unix, SGI IRIX, Siemens Reliant Unix, and others.

There may be significant advantages to carrying out the functions of portal 400 on multiple computer platforms in this manner, such as lower costs of deployment, database switching, or changes to enterprise applications, and/or more effective firewalls. Other configurations, however, are possible. For example, as is well known to those of ordinary skill in the relevant art, so-called two-tier or N-tier-architectures are possible rather than the three-tier server-side component architecture represented by FIG. 5. See, for example, E. Roman, Mastering Enterprise JavaBeans™ and the Java™2 Platform (John Wiley & Sons, Inc., NY, 1999) and J. Schneider and R. Arora, Using Enterprise Java™ (Que Corporation, Indianapolis, 1997), both of which are hereby incorporated by reference in their entireties for all purposes.

It will be understood that many hardware and associated software or firmware components that may be implemented in a server-side architecture for Internet commerce are not shown in Figure S. Components to implement one or more firewalls to protect data and applications, uninterruptable power supplies, LAN switches, web-server routing software, and many other components are not shown. Similarly, a variety of computer components customarily included in server-class computing platforms, as well as other types of computers, will be understood to be included but are not shown. These components include, for example, processors, memory units, input/output devices, buses, and other components noted above with respect to user computer 103. Those of ordinary skill in the art will readily appreciate how these and other conventional components may be implemented.

The functional elements of portal 400 also may be implemented in accordance with a variety of software facilitators and platforms (although it is not precluded that some or all of the functions of portal 400 may also be implemented in hardware or firmware). Among the various commercial products available for implementing e-commerce web portals are BEA WebLogic from BEA Systems, which is a so-called “middleware” application. This and other middleware applications are sometimes referred to as “application servers,” but are not to be confused with application server 520, which is a computer. The function of these middleware applications generally is to assist other software components (such as managers 512, 522, or 532) to share resources and coordinate activities. The goals include making it easier to write, maintain, and change the software components; to avoid data bottlenecks; and prevent or recover from system failures. Thus, these middleware applications may provide load-balancing, fail-over, and fault tolerance, all of which features will be appreciated by those of ordinary skill in the relevant art.

Other development products, such as the Java™ 2 platform from Sun Microsystems, Inc. may be employed in portal 400 to provide suites of applications programming interfaces (API's) that, among other things, enhance the implementation of scalable and secure components. The platform known as J2EE (Java™2, Enterprise Edition), is configured for use with Enterprise JavaBeans™, both from Sun Microsystems. Enterprise JavaBeans™ generally facilitates the construction of server-side components using distributed object applications written in the Java™ language. Thus, in one implementation, the functional elements of portal 400 may be written in Java and implemented using J2EE and Enterprise JavaBeans™. Various other software development approaches or architectures may be used to implement the functional elements of portal 400 and their interconnection, as will be appreciated by those of ordinary skill in the art.

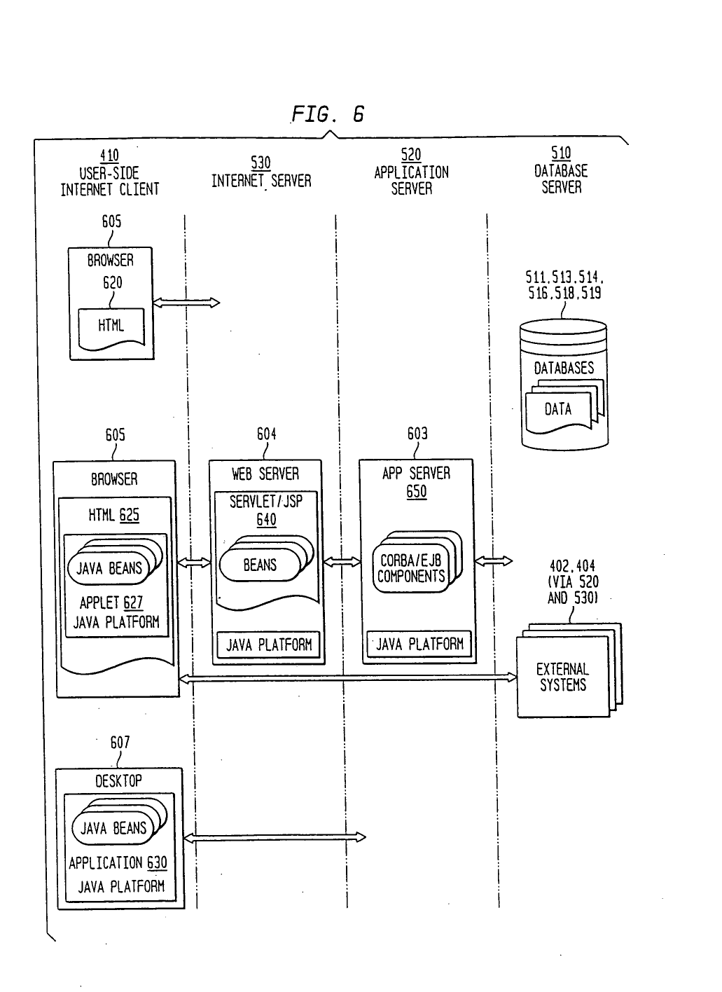

One implementation of these platforms and components is shown in FIG. 6. FIG. 6 is a simplified graphical representation of illustrative interactions between user-side internet client 410 on the user side and input and output managers 532 and 534 of Internet server 530 on the portal side, as well as communications among the three tiers (servers 510, 520, and 530) of portal 400. Browser 605 on client 410 sends and receives HTML documents 620 to and from server 530. HTML document 625 includes applet 627. Browser 605, running on user computer 103, provides a run-time container for applet 627. Functions of managers 532 and 534 on server 530, such as the performance of GUI operations, may be implemented by servlet and/or JSP 640 operating with a Java™ platform. A servlet engine executing on server 530 provides a runtime container for servlet 640. JSP (Java Server Pages) from Sun Microsystems, Inc. is a script-like environment for GUI operations; an alternative is ASP (Active Server Pages) from the Microsoft Corporation. App server 650 is the middleware product referred to above, and executes on application server 520. EJB (Enterprise JavaBeans™ is a standard that defines an architecture for enterprise beans, which are application components. CORBA (Common Object Request Broker Architecture) similarly is a standard for distributed object systems, i.e., the CORBA standards are implemented by CORBA-compliant products such as Java™ IDL. An example of an EJB-compliant product is WebLogic, referred to above. Further details of the implementation of standards, platforms, components, and other elements for an Internet portal and its communications with clients, are well known to those skilled in the relevant art.

As noted, one of the functional elements of portal 400 is input manager 532. Manager 532 receives a set, i.e., one or more, of probe-set identifiers from user 101 over Internet 499. Manager 532 processes and forwards this information to user-service manager 522. These functions are performed in accordance with known techniques common to the operation of Internet servers, also commonly referred to in similar contexts as presentation servers. Another of the functional elements of portal 400 is output manager 534. Manager 534 provides information assembled by user-service manager 522 to user 101 over Internet 499, also in accordance with those known techniques, aspects of which were described above in relation to FIG. 6. The information assembled by manager 522 is represented in FIG. 5 as data 524, labeled “integrated genomic and/or product web pages responsive to user request.” The data is integrated in the sense, among other things, that it is based, at least in part, on the specification by user 101 of probe-set identifiers and thus has common relationships to the genes and/or EST's corresponding to those identifiers. The presentation by manager 534 of data 524 may be implemented in accordance with a variety of known techniques. As some examples, data 524 may include HTML or XML documents, email or other files, or data in other forms. The data may include Internet URL addresses so that user 101 may retrieve additional HTML, XML, or other documents or data from remote sources.

Portal 400 further includes database manager 512. In the illustrated embodiment, database manager 512 coordinates the storage, maintenance, supplementation, and all other transactions from or to any of local databases 511, 513, 514, 516, and 518. Manager 512 may undertake these functions in cooperation with appropriate database applications such as the Oracle® 8.0.5 database management system.

In some implementations, manager 512 periodically updates local genomic database 518. The data updated in database 518 includes data related to genes or EST's that correspond with one or more probe sets. The probe sets may be those used or designed for use on any microarray product, and/or that are expected or calculated to be used in microarray products of any manufacturer or researcher. For example, the probe sets may include all probe sets synthesized on the line of stocked GeneChip® probe arrays from Affymetrix, Inc., including its Arabidopsis Genome Array, CYP450 Array, Drosophila Genome Array, E. coli Genome Array, GenFlex™ Tag Array, HIV PRT Plus Array, HuGeneFL Array, Human Genome U95 Set, HuSNP Probe Array, Murine Genome U74 Set, P53 Probe Array, Rat Genome U34 Set, Rat Neurobiology U34 Set, Rat Toxicology U34 Array, or Yeast Genome S98 Array. The probe sets may also include those synthesized on custom arrays for user 101 or others. However, the data updated in database 518 need not be so limited. Rather, it may relate to any number of genes or EST's. Types of data that may be stored in database 518 are described below in relation to the operations of manager 522 in directing the periodic collection of this data from remote sources providing the locally maintained data in database 518 to users.

Database 516 includes data of a type referred to above in relation to database application 230, i.e., data that associates probe sets with their corresponding gene or EST and their identifiers. Database 516 may also include SIF's, and other library data. User-service manager 522 may provide database manager 512 from time to time with update information regarding library and other data. In some cases, this update information will be provided by the owners or managers of proprietary information, although this information may also be made available publicly, as on a web site, for uploading.

Information for storage by manager 512 in local products database 514 may similarly be provided by vendors, distributors, or agents, or obtained from public sources such as web sites. A wide variety of product-related information may be included in database 514, examples of which include availability, pricing, composition, suitability, or ordering data. The information may relate to a wide variety of products, including any type of biological device or substance, or any type of reagent that may be used with a biological device or substance. To provide just a few examples, the device, substance, or reagent may be an oligonucleotide, probe array, clone, antibody, or protein. The data stored in database 514 may also include links, such as Internet URL addresses, to remote sites where product data is available, such as vendors' web sites.

Database 511 includes information relating probe-set identifiers to the sequences of the probes. This information may be provided by the manufacturer of the probes, the researchers who devise probes for spotted arrays or other custom arrays, or others. Moreover, the application of portal 400 is not limited to probes arranged in arrays. As noted, probes may be immobilized on or in beads, optical fibers, or other substrates or media. Thus, database 511 may also include information regarding the sequences of these probes.

Database 519 includes information about users and their accounts for doing business with or through portal 400. Any of a variety of account information, such as current orders, past orders, and so on, may be obtained from users, all as will be readily apparent to those of ordinary skill in the art. Also, information related to users may be developed by recording and/or analyzing the interactions of users with portal 400, in accordance with known techniques used in e-commerce. For example, user-service manager 522 may take note of users' areas of genomic interest, their purchase or product-inquiry activities, the frequency of their accessing of various services, and so on, and provide this information to database manager 512 for storage or update in database 519.

Another functional element of portal 400 is user-service manager 522. Manager 522 may periodically cause database manager 512 to update local genomic database 518 from various sources, such as remote databases 402. For example, according to any chronological schedule (e.g., daily, weekly, etc.), manager 522 may, in accordance with known techniques, initiate searches of remote databases 402 by formulating appropriate queries, addressed to the URL's of the various databases 402, or by other conventional techniques for conducting data searches and/or retrieving data or documents over the Internet. These search queries and corresponding addresses may be provided in a known manner to output manager 534 for presentation to databases 402. Input manager 532 receives replies to the queries and provides them to manager 522, which then provides them to database manager 512 for updating of database 518, all in accordance with any of a variety of known techniques for managing information flow to, from, and within an Internet site.

Portal application manager 526 manages the administrative aspects of portal 400, possibly with the assistance of a middleware product such as an applications server product. One of these administrative tasks may be the issuance of periodic instructions to manager 522 to initiate the periodic updating of database 518 just described. Alternatively, manager 522 may self-initiate this task. It is not required that all data in database 518 be updated according to the same periodic schedule. Rather, it may be typical for different types of data and/or data from different sources to be updated according to different schedules. Moreover, these schedules may be changed, and need not be according to a consistent schedule. That is, updating for particular data may occur after a day, then again after 2 days, then at a different period that may continue to vary. Numerous factors may influence the determination by manager 526 or manager 522 to maintain or vary these periods, such as the response time from various remote databases 402, the value and/or timeliness of the information in those databases, cost considerations related to accessing or licensing the databases, the quantity of information that must be accessed, and so on.

In some implementations, manager 522 constructs from data in local genomic database 518 a set of data related to genes or EST's corresponding to the set of probe-set identifiers selected by user 101. The user selection may be forwarded to manager 522 by input manager 532 in accordance with known techniques. Manager 522, also in accordance with known techniques, obtains the data from database 518 by forming appropriate queries, such as in one of the varieties of SQL language, based on the user selection. Manager 522 then forwards the queries to database manager 512 for execution against database 518.

As noted, various types of data may be accessed from remote databases 402 and maintained in local genomic database 518 in this manner. Examples include sequence data, exonic structure or location data, splice-variants data, marker structure or location data, polymorphism data, homology data, protein-family classification data, pathway data, alternative-gene naming data, literature-recitation data, and annotation data. Many other examples are possible. Also, genomic data not currently available but that becomes available in the future may be accessed and locally maintained as described herein. Examples of remote databases 402 currently suitable for accessing in the manner described include GenBank, GenBank New, SwissProt, GenPept, DB EST, Unigene, PIR, Prosite, PFAM, Prodom, Blocks, PDB, PDBfinder, EC Enzyme, Kegg Pathway, Kegg Ligand, OMIM, OMIM Map, OMIM Allele, DB SNP, and PubMed. Hundreds of other databases currently exist that are suitable, and thus this list is merely illustrative.

Moreover, local genomic database 518 may also be supplemented with data obtained or deduced (by user-service manager 522) from other of the local databases serviced by database manager 512. In particular, although local products database 514 is shown for convenience of illustration as separate from database 518, it may be the same database. Alternatively, or all or part of the data in database 514 may be duplicated in, or accessible from, database 518.

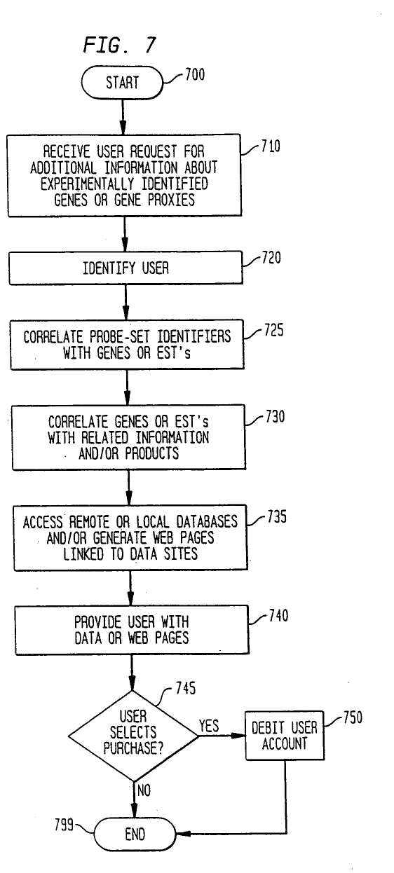

More specific examples are now provided of how user service manager 522 may receive and respond to requests from user 101 for genomic information and for product information and/or ordering. These examples are described in relation to FIGS. 7, 8 and 9.

FIG. 7 is a flow chart representing an illustrative method by which the illustrated embodiment of portal 400 may respond to a user's request for genomic or product information. In accordance with step 710 of this example, input manager 532 receives from client 410 over Internet 499 a request by user 101 for data. This request may, for instance, include an HTML or XML document that includes user 101's selection of certain probe-set identifiers. As noted, the probe-set identifiers may be a number, name, accession number, symbol, graphical representation, or nucleotide or other sequence, as non-limiting examples. In some cases, user 101 may make this selection by employing one or more of analysis applications 199A to select probe-set identifiers (e.g., by drawing a loop around dots, as noted above) and then activating communication with portal 400 by any of a variety of known techniques such as right-clicking a mouse. The request may also, in accordance with any of a variety of known techniques, specify whether user 101 is interested in genomic and/or product data, as well as details regarding the type of data that is desired. For instance, user 101 may select categories of products, names of vendors or products, and so on from pull-down menus. Manager 532 provides user 101's request to user service manager 522, as described above.

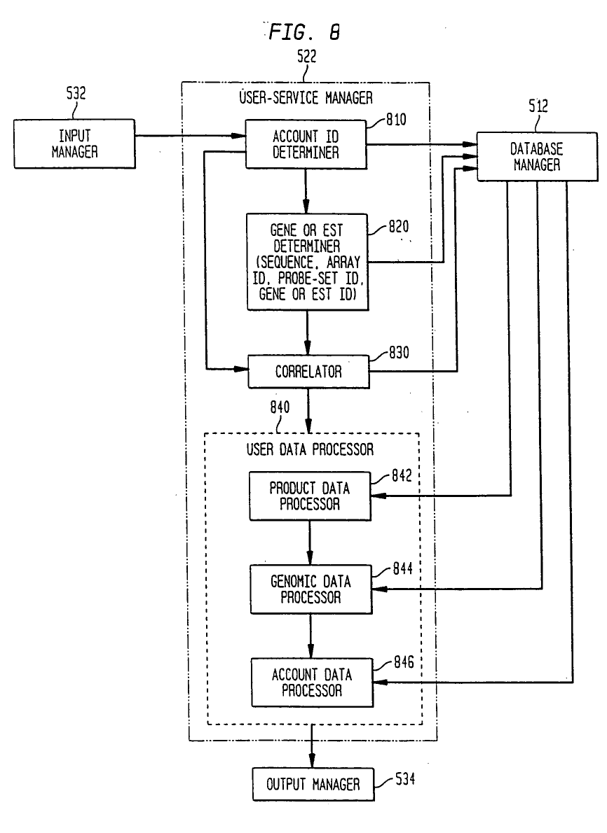

In accordance with step 720, user-service manager 522 initiates an identification of user 101. FIG. 8 is a block diagram showing the functional elements of manager 522 in greater detail, including account ID determiner 822 that, in this illustrative implementation, undertakes the task of identifying user 101. Determiner 822 may employ any of various known techniques to obtain this information, such as the use of cookies or the extraction from the user's request of an identification number entered by the user. Determiner 810, through database manager 512, may compare the user's identification with entries in user account database 519 to further identify user 101. In other implementations, the identity of user 101 need not be obtained, although statistics or information regarding user 101's request may be recorded, as noted above.

In accordance with step 725, user-service manager 522 formulates an appropriate query (using, for example, a version of the SQL language) for correlating probe-set identifiers with corresponding genes or EST's. Gene or EST determiner 820 is the functional element of manager 522 that illustratively executes this task. Determiner 820 forward the query to database manager 512. If the probe-set identifiers provided by user 101 include sequence information, then the query may seek from database 511, and/or from SIF information in database 516, the identity of the one or more probe sets having a corresponding (e.g., similar in biological significance) sequence. If the probe-set identifiers include-names or numbers (e.g., accession numbers), then the query may seek the identity of the probe sets from database 516 that, as noted, includes data that associates names, numbers, and other probe-set identifiers with corresponding genes or EST's. User 101 may also have locally employed database application 230 to obtain this information, and included it in the information request in accordance with known techniques. In this case, step 725 need not be performed.

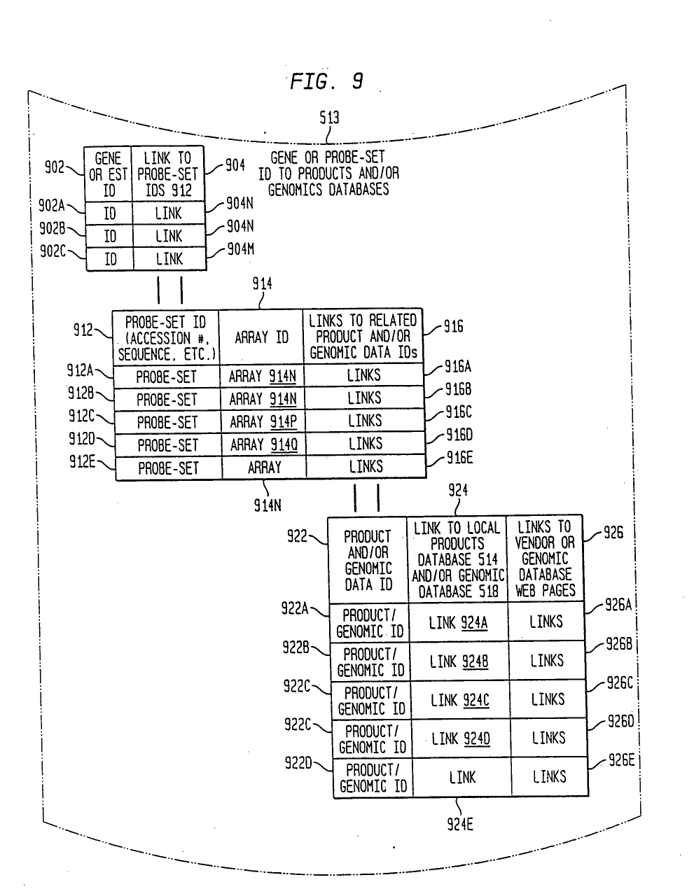

As indicated in step 730, user-service manager 522 may then correlate the indicated genes and/or EST's with genomic information and/or product information. The performance of this task is undertaken by correlator 830 in the illustrated example. In one of many possible implementations, correlator 830 formulates a query via database manager 512 to database 513 in order to obtain links to appropriate information in local products database 514 and/or local genomic database 518. FIG. 9 is a simplified graphical representation of database 513. Those of ordinary skill in the art will appreciate that this representation is provided for purposes of clarity of illustration, and that many other implementations are possible. In one aspect of an appropriate query to database 513, which is assumed for illustration to be a relational database, a gene or EST accession number 902 is associated with a link 904 to probe-set ID's 912. As indicated in FIG. 9 by the association of both ID 902A and 902B to the same link 904N, multiple genes and/or EST's may be associated with the same probe-set ID. The information used to establish these associations is similar to that provided in database 516, as noted above, and the links may thus be predetermined or dynamically determined using database 516.

In other implementations, correlator 830 simply correlates one or more gene or EST identifiers, such as accession numbers, with products, such as biological products. These implementations are indicated in FIG. 8 by the arrow directly from determiner 810 (which is optional) directly to correlator 830. The correlation may be accomplished according to any of a variety of conventional techniques, such as by providing a query to local products database 514, remote pages 404, and/or remote databases 402. These queries may be indexed or keyed by categories, types, names, or vendors of products, such as may be appropriate, for example, in examining look-up tables, relational databases, or other data structures. In addition, the query may, in accordance with techniques known to those of ordinary skill in the relevant art, search for products, product web pages, or other product data sources that are logically or syntactically associated with the gene or EST identifier(s). The results of the query may then be provided by output manager 534 to user 101, such as over Internet 499 to client 410.

Following the appropriate links 904 to probe-set ID's 912, one or more links 916 to related products and/or genomic data may be obtained. For example, link 904N may link to probe-set 912C, which is associated with links 916C to related product and/or genomic data. The information used to establish this association may be predetermined based on expert input and/or computer-implemented analysis (e.g., statistical and/or by an adaptive system such as a neural network) of the nature of inquiries by users. For example, it may be observed or anticipated (by humans or computers, as noted) that users conducting gene expression experiments resulting in the identification of certain genes may wish to use antibodies against the genes to conduct follow-on protein level experiments. The association between the genes and the appropriate antibodies may be stored in an appropriate database, such as database 516. Links 916C may thus include links to product or genomic data identifiers that identify links to data about the appropriate antibodies (for example, a link to product/genomic ID 922A), to catalogues of antibodies generally (e.g., ID 922B), or to a probe array specifically designed for detecting alternatively spliced forms of the genes of interest (e.g., ID 922C). It is assumed for illustrative purposes that, in a particular aspect of this example, link 916C leads to ID 922C. Information about the availability of splice-variant probe arrays may be predetermined by the contents of links 926. For example, links 926D (associated with ID 922C, as shown) may be stored Internet and/or database-query URL's leading to vendor web pages, local products database 514, and/or local genomic database 518. Also, the content of links 926D may be dynamically determined by query of databases 514 or 518 or of remote data sources such as databases 402 or web pages 404. These and similar processes are represented by step 735 of FIG. 7.

As will now be appreciated by those of ordinary skill in the art, numerous variations and alternative implementations of this illustrative arrangement of database 513 are possible. For example, probe-set identification data may be linked to array identifiers (such as array ID 914), which may then be associated with links 916. As another of many possible examples, gene or EST accession numbers may be linked directly to product and/or genomic data ID 922 or, even more directly, to links 926. Implementations such as the illustrated one provide opportunities for making broad associations based on a more narrow inquiry by a user. For instance, a user may select only one probe-set identifier, but that identifier may be linked to multiple genes and/or EST's, which may be linked to multiple products or genomic data. In another example, link 926D may include a link to local genomic database 518. Based on the probe-set identifiers, gene or EST accession numbers, sequence information, or other data provided by or deduced from user 101's inquiry, database 518 may be searched for associated data in accordance with known query and/or search techniques.

Returning now to FIG. 7 and step 740 in particular, data returned in accordance with the query posed by correlator 830 is provided to either product data processor 842, genomic data processor 844, or both, as appropriate in view of the nature of the returned data. The functions of processors 842 and 844 are shown as separated for convenience of illustration, but it need not be so. Processors 842 and 844 apply any of a variety of known presentation or data transfer techniques to prepare graphical user interfaces, files for transfer, and other forms of data. This processed data is then provided to output manager 534 for transmission to client 410.

In some implementations, user 101 may respond to the data thus transmitted by indicating a desire to purchase a product or receive further information. A request for further information may be processed in a manner similar to that described above with respect to FIG. 7. If user 101 indicates a desire to purchase a product (see decision element 745), the indicated product may be prepared for shipment or otherwise processed, and the user's account may be adjusted, in accordance with known techniques for conducting e-commerce. As one of many alternative implementations, user-service manager 522 may notify the product vendor of user 101's order and the vendor may ship, or order the shipment of, the product. Manager 522 may then note, in one aspect of this implementation, that a fee should be collected from the vendor for the referral.

In some implementations of portal 400, user 101 may provide to portal 400 (e.g., via client 410, Internet 499, and input manager 532) one or more gene or EST ascension numbers or other gene or EST identifiers. Alternatively, or in addition, user 101 may provide to portal 400 one or more probe-set identifiers. User 101 may obtain the gene, EST, and/or probe-set identifier from a public source, from notations user 101 has taken as a result of experiments with a probe array or otherwise, from a list of genes or EST's having corresponding probes on a probe array, or from any other source or obtained in any other manner. Input manager 532 receives the one or more gene, EST, or probe-set identifiers and provides it or them to user-service manager 522, which formulates a query to database manager 512. In accordance with known query techniques and formats, the query seeks information from local products database 514 of product information related to the gene, EST, and/or probe-set identifiers. For this purpose, local products database 514 may be indexed, or otherwise searchable, for products based or keyed on any one or more of gene, EST, and/or probe-set identifiers. Some implementations may include, according to known techniques, similarity matching of a gene, EST, or probe-set identifier if, for example, all or part of a gene, EST, SFI (corresponding to the probe-set identifier) sequence is submitted. Also, a name-association function, in accordance with known techniques such as look-up tables, may be performed so that alternative names or forms of a gene, EST, or probe-set identifier may be found and used in the product data inquiry. In addition, in some implementations, manager 522 may initiate a remote data search of remote databases 402 and/or remote vendor web pages 404, in accordance with known Internet search techniques, to obtain product information from remote sources. These searches may be based, for example, on product categories or vendors associated in local products database 514 with products, categories, or vendors associated with the gene, EST, or probe-set identifier provided by user 101. Manager 522 may provide product data corresponding to the gene, EST, and/or probe-set identifier, obtained from local products database 514 and/or remote pages or databases 404 or 402, and provide this product data to user 101 via output manager 534. For example, this product data may be included in web pages 524. In some of these implementations, portal 400 thus provides a system for providing product data, typically biological product data. The system includes input manager 532 that receives from user 101 one or more of a gene, EST, and/or probe-set identifier; user-service manager 522 that correlates the gene, EST, and/or probe-set identifier with one or more product data and that causes (e.g., via database manager 512) the product data to be obtained either locally from, e.g., database 514 or, in some implementations, remotely from, e.g., pages 404 or databases 402; and output manager 534 that provides the product data to user 101.

Similarly, a method is provided for providing biological product data, including the steps of: receiving from user 101 any one or more of a gene, EST, and/or probe-set identifier; correlating the gene, EST, and/or probe-set identifier with one or more product data; causing the product data to be obtained either locally from, e.g., database 514 and/or remotely from, e.g., pages 404 or databases 402; and providing the product data to user 101.

As indicated above, functional elements of portal 400 may be implemented in hardware, software, firmware, or any combination thereof. In the embodiment described above, it generally has been assumed for convenience that the functions of portal 400 are implemented in software. That is, the functional elements of the illustrated embodiment comprise sets of software instructions that cause the described functions to be performed. These software instructions may be programmed in any programming language, such as Java, Perl, C++, another high-level programming language, low-level languages, and any combination thereof. The functional elements of portal 400 may therefore be referred to as carrying out “a set of genomic web portal instructions,” and its functional elements may similarly be described as sets of genomic web portal instructions for execution by servers 510, 520, and 530.

In some embodiments, a computer program product is described comprising a computer usable medium having control logic (computer software program, including program code) stored therein. The control logic, when executed by a processor, causes the processor to perform functions of portal 400 as described herein. In other embodiments, some such functions are implemented primarily in hardware using, for example, a hardware state machine. Implementation of the hardware state machine so as to perform the functions described herein will be apparent to those skilled in the relevant arts.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments. Also, the functions of several elements may, in alternative embodiments, be carried out by fewer, or a single, element.

For example, for purposes of clarity the functions of user-service manager 522 are described as being implemented by the functional elements shown in FIG. 8. However, manager 522 need not be divided into these, or other, distinct functional elements. Similarly, operations of a particular functional element that are described separately for convenience need not be carried out separately. For example, some or all of the functions of product data processor 842 could be implemented by genomic data processor 844, and vice versa. Similarly, in some embodiments, any functional element may perform fewer, or different, operations than those described with respect to the illustrated embodiment. Also, functional elements shown as distinct for purposes of illustration may be incorporated within other functional elements in a particular implementation. For example, the functions of processors 842 and 844 could be ascribed to a single functional element. Similarly, some or all of the functions of database manager 512 could be carried out by user-service manager 522, and/or by input manager 532.

Also, the sequencing of functions or portions of functions generally may be altered. For example, the functions of account ID determiner 810 may be carried out after those of user data processor 840. The flow of data and control in FIG. 8 in this regard thus is exemplary only. Similarly, the method steps shown in FIG. 7 need not always be carried out in the order suggested by the illustrative example of that figure. For instance, method step 720 of identifying the user could be carried out after that of steps 725, 730, or 735.

Certain functional elements, files, data structures, and so on, may be described in the illustrated embodiments as located in system memory 120 of computer 100 or generally in servers 510, 520, or 530. In other embodiments, however, they may be located on, or distributed across, computer systems or other platforms that are co-located and/or remote from each other. For example, any one or more of data files or data structures 511, 513, 514, 516, or 518, shown in FIG. 5 as co-located on and “local” to server 510, may be located in a computer system or systems remote from server 510. In those cases, the operations of database manager 512 with respect to these data files or data structures may be carried out over a network or by any of numerous other known means for transferring data and/or control to or from a remote location.

In addition, it will be understood by those skilled in the relevant art that control and data flows between and among functional elements and various data structures may vary in many ways from the control and data flows described above. More particularly, intermediary functional elements (not shown) may direct control or data flows, and the functions of various elements may be combined, divided, or otherwise rearranged to allow parallel processing or for other reasons. Also, intermediate data structures or files may be used and various described data structures or files may be combined or otherwise arranged. Numerous other embodiments, and modifications thereof, are contemplated as falling within the scope of the present invention as defined by appended claims and equivalents thereto.

1-52. (Cancelled)

53. A system for providing data related to one or more genes or EST's, wherein each gene or EST has at least one corresponding probe set identified by a probe-set identifier and capable of enabling detection of a biological molecule, comprising:
a database manager constructed and arranged to periodically update a local genomic database comprising data related to the genes or EST's;
an input manager constructed and arranged to receive from a user a selection of a first set of one or more of the probe-set identifiers;
a user-service manager constructed and arranged to construct from the local genomic database a first set of data related to genes or EST's corresponding to the first set of probe-set identifiers; and
an output manager constructed and arranged to provide the first set of data to the user.

54. The system of claim 53, wherein:
the first set of probe-set identifiers identify probe sets that are capable of enabling the detection of a biological molecule that consists of nucleic acid.

55. The system of claim 53, wherein:
the first set of probe-set identifiers identify probe sets that are capable of enabling the detection of a biological molecule that consists of mRNA transcripts of corresponding genes.

56. The system of claim 53, wherein:
the database manager updates the local genomic database according to a chronological period.

57. The system of claim 56, wherein:
the chronological period is predetermined.

58. The system of claim 56, wherein:
the chronological period is greater than about ten hours and less than about ten days.

59. The system of claim 53, wherein:
the database manager periodically updates the local genomic database with update data consisting of any combination of one or more of sequence data, exonic structure or location data, splice-variants data, marker structure or location data, polymorphism data, homology data, protein-family classification data, pathway data, alternative-gene naming data, literature-recitation data, or annotation data.

60. The system of claim 53, wherein:
the database manager periodically updates the local genomic database with update data from one or more remote databases.

61. The system of claim 60, wherein:
the updating from one or more remote databases comprises updating over the Internet.

62. The system of claim 61, wherein:
the remote databases consist of any combination of one or more of GenBank, GenBank New, SwissProt, GenPept, DB EST, Unigene, PIR, Prosite, PFAM, Prodom, Blocks, PDB, PDBfinder, EC Enzyme, Kegg Pathway, Kegg Ligand, OMIM, OMIM Map, OMIM Allele, DB SNP, and PubMed.

63. The system of claim 53, wherein:
the input manager is constructed and arranged to dynamically receive the user-initiated selection.

64. The system of claim 53, wherein:
the first group comprises all or part of a second set of one or more probe-set identifiers of probe sets that have enabled detection of the expression or differential expression of their corresponding genes or EST's.

65. The system of claim 64, wherein:
the probe sets identified by the second set of probe-set identifiers are disposed on one or more probe arrays.

66. The system of claim 65, wherein:
the probe arrays include a GeneChip® probe array.

67. The system of claim 65, wherein:
the probe sets include a single spotted probe;
the probe-set identifiers include a spotted probe identifier that identifies the single spotted probe; and
the probe arrays include a spotted array that includes the single spotted probe.

68. The system of claim 67, wherein:
the single spotted probe includes an oligonucleotide.

69. The system of claim 64, wherein:
the user includes a remote user, and
the input manager receives the remote user's selection over a network.

70. The system of claim 69, wherein:
the network includes the Internet.

71. The system of claim 53, wherein:
the user includes a remote user, and
the output manager provides the first set of data to the user over a network.

72. The system of claim 71, wherein:
the network includes the Internet.

73. The system of claim 53, wherein:
at least one of the probe-set identifiers comprises a gene identifier of the gene corresponding to the probe-set identifier.

74. The system of claim 73, wherein:
the gene identifier comprises an accession number.

75-102. (Cancelled)
